(19) United States
(12) Patent Application Publication
(10) Pub. No.: US 2020/0263223 A1
(43) Pub. Date: Aug. 20, 2020

(54) MICROBIOME HEALTH INDEX

(71) Applicant: REBIOTIX, INC., Roseville, MN (US)

(72) Inventors: KENNETH F. BLOUNT, Guilford, CT (US); WILLIAM DOUGLAS SHANNON, St. Louis, MO (US); ELENA DEYCH, St. Louis, MO (US); COURTNEY JONES, Fridley, MN (US); GREGORY J. FLUET, Minneapolis, MN (US)

(73) Assignee: REBIOTIX, INC., Roseville, MN (US)

(21) Appl. No.: 16/651,469

(22) PCT Filed: Oct. 12, 2018
(86) PCT No.: PCT/US2018/055742

§ 371 Date: Mar. 27, 2020

Related U.S. Application Data
(60) Provisional application No. 62/571,907, filed on Oct. 13, 2017.

(51) Int. Cl. C12Q 1/06

(52) U.S. Cl. CPC C12Q 1/06

(57) ABSTRACT

Methods for assessing microbiota are disclosed. An example method for assessing microbiota may include obtaining a fecal sample from a patient, quantifying the relative abundance of bacteria from a selected group of taxonomic classes in the fecal sample, calculating a microbiome health index based on the relative abundance of bacteria from the selected group of taxonomic classes, and correlating the microbiome health index with a medical condition of the patient.

### CROSS-REFERENCE TO RELATED APPLICATIONS

This application claims priority under 35 U.S.C. § 119 to U.S. Provisional Application Ser. No. 62/571,907, filed Oct. 13, 2017, the entirety of which is incorporated herein by reference.

### TECHNICAL FIELD

The present disclosure pertains to the microbiome of the intestinal tract. More particularly, the present disclosure pertains to balancing the microbiome, treating the microbiome, assessing a medical disorder by examining the microbiome, and/or assessing a medical treatment by examining the microbiome.

### BACKGROUND

A wide variety of medical disorders and/or conditions may be associated with the microbiome of the intestinal tract.

### BRIEF SUMMARY

A method for assessing microbiota is disclosed. The method comprises: obtaining a fecal sample; quantifying the relative abundance of bacteria from a selected group of taxonomic classes in the fecal sample; calculating a microbiome health index based on the relative abundance of bacteria from the selected group of taxonomic classes; and considering the calculated microbiome health index to assess the microbiota of the fecal sample.

Alternatively or additionally to any of the embodiments above, the fecal sample may come from one person or from multiple persons.

Alternatively or additionally to any of the embodiments above, the selected group of taxonomic classes includes bacteria from the class Bacteroidia.

Alternatively or additionally to any of the embodiments above, the selected group of taxonomic classes includes bacteria from the class Clostridia.

Alternatively or additionally to any of the embodiments above, the selected group of taxonomic classes includes bacteria from the class Gammaproteobacteria.

Alternatively or additionally to any of the embodiments above, the selected group of taxonomic classes includes bacteria from the class Bacilli.

Alternatively or additionally to any of the embodiments above, calculating a microbiome health index includes determining a first sum of relative bacterial abundance, the first sum of bacterial abundance being equal to the sum of a first relative abundance of a first taxonomic class of bacteria and a second relative abundance of a second taxonomic class of bacteria; determining a second sum of relative bacterial abundance, the second sum of bacterial abundance being equal to the sum of a third relative abundance of a third taxonomic class of bacteria and a fourth relative abundance of a fourth taxonomic class of bacteria; and dividing the first sum of relative bacterial abundance by the second sum of relative bacterial abundance.

Alternatively or additionally to any of the embodiments above, the first relative abundance of bacteria is the relative abundance of bacteria from the taxonomic class Bacteroidia in the fecal sample.

Alternatively or additionally to any of the embodiments above, the second relative abundance of bacteria is the relative abundance of bacteria from the taxonomic class Clostridia in the fecal sample.

Alternatively or additionally to any of the embodiments above, the third relative abundance of bacteria is the relative abundance of bacteria from the taxonomic class Gammaproteobacteria in the fecal sample.

Alternatively or additionally to any of the embodiments above, the fourth relative abundance of bacteria is the relative abundance of bacteria from the taxonomic class Bacilli in the fecal sample.

A method for assessing a medical treatment is disclosed. The method comprises: treating a patient for a medical disorder; obtaining a post-treatment fecal sample from the patient after the treatment; calculating a microbiome health index based on the relative abundance of bacteria from a selected group of taxonomic classes in the post-treatment fecal sample; and considering the microbiome health index to evaluate the effectiveness of treating the patient for the medical disorder.

Alternatively or additionally to any of the embodiments above, the method for assessing a medical treatment further comprises quantifying the relative abundance of bacteria from a selected group of taxonomic classes in the fecal sample.

Alternatively or additionally to any of the embodiments above, the method for assessing a medical treatment further comprises obtaining a pre-treatment fecal sample from the patient prior to the treatment and calculating another microbiome health index based on the relative abundance of bacteria from the selected group of taxonomic classes in the pre-treatment fecal sample. And further in some embodiments, the method further comprises comparing the microbiome health index of the pre-treatment fecal sample to the microbiome health index of the post-treatment fecal sample to evaluate the effectiveness of the treatment.

Alternatively or additionally to any of the embodiments above, the selected group of taxonomic classes includes bacteria from the class Bacteroidia.

Alternatively or additionally to any of the embodiments above, the selected group of taxonomic classes includes bacteria from the class Clostridia.

Alternatively or additionally to any of the embodiments above, the selected group of taxonomic classes includes bacteria from the class Gammaproteobacteria.

Alternatively or additionally to any of the embodiments above, the selected group of taxonomic classes includes bacteria from the class Bacilli.

Alternatively or additionally to any of the embodiments above, calculating a microbiome health index includes determining a first sum of relative bacterial abundance, the first sum of bacterial abundance being equal to the sum of a first relative abundance of a first taxonomic class of bacteria and a second relative abundance of a second taxonomic class of bacteria; determining a second sum of relative bacterial abundance, the second sum of bacterial abundance being equal to the sum of a third relative abundance of a third taxonomic class of bacteria and a fourth relative abundance of a fourth taxonomic class of bacteria; and dividing the first sum of relative bacterial abundance by the second sum of relative bacterial abundance.

Alternatively or additionally to any of the embodiments above, the first relative abundance of bacteria is the relative abundance of bacteria from the taxonomic class Bacteroidia in the fecal sample.

Alternatively or additionally to any of the embodiments above, the second relative abundance of bacteria is the relative abundance of bacteria from the taxonomic class Clostridia in the fecal sample.

Alternatively or additionally to any of the embodiments above, the third relative abundance of bacteria is the relative abundance of bacteria from the taxonomic class Gammaproteobacteria in the fecal sample.

Alternatively or additionally to any of the embodiments above, the fourth relative abundance of bacteria is the relative abundance of bacteria from the taxonomic class Bacilli in the fecal sample.

Alternatively or additionally to any of the embodiments above, treating a patient with a medical disorder includes treating the patient for a gastrointestinal disorder.

Alternatively or additionally to any of the embodiments above, treating a patient with a medical disorder includes administering a microbiota restoration therapy composition to the patient.

Alternatively or additionally to any of the embodiments above, the microbiota restoration therapy composition includes a processed fecal sample and a diluent including polyethylene glycol at a concentration of 30-90 g/L in saline.

Alternatively or additionally to any of the embodiments above, the microbiota restoration therapy composition has a microbiome health index (calculated based on the relative abundance of bacteria from the selected group of taxonomic classes in the composition) that is higher than the microbiome health index of the pre-treatment and the post-treatment fecal samples.

Alternatively or additionally to any of the embodiments above, the post-treatment fecal samples is taken after the patient was treated for the medical disorder for a period of time ranging from 7 to 30 days.

Alternatively or additionally to any of the embodiments above, the post-treatment fecal samples is taken after the patient was treated for the medical disorder for 7 days or more.

A method for medical treatment is disclosed. The method comprises: administering a microbiota restoration therapy composition to a patient with a medical disorder; obtaining a fecal sample from the patient at least 7 days after administering the microbiota restoration therapy composition to the patient; quantifying the relative abundance of bacteria from a selected group of taxonomic classes in the fecal sample; calculating a microbiome health index based on the relative abundance of bacteria from the selected group of taxonomic classes in order to assess the effectiveness of treatment; and wherein the selected group of taxonomic classes include Bacteroidia, Clostridia, Gammaproteobacteria, and Bacilli.

A method for assessing microbiota of a patient is disclosed. The method comprises: obtaining a fecal sample from a patient; calculating a microbiome health index based on the relative abundance of bacteria from a selected group of taxonomic classes; and assessing the microbiota of the patient based on the calculated microbiome health index.

Alternatively or additionally to any of the embodiments above, the selected group of taxonomic classes include Bacteroidia, Clostridia, Gammaproteobacteria, and Bacilli.

A method for assessing a medical treatment is disclosed. The method comprises: treating a patient for a medical disorder; a period of time after treating the patient for the medical disorder, obtaining a fecal sample from a patient; calculating a microbiome health index based on the relative abundance of bacteria from a selected group of taxonomic classes in the fecal sample; and considering the calculated microbiome health index to evaluate the success of treating the patient for the medical disorder.

Alternatively or additionally to any of the embodiments above, the selected group of taxonomic classes include Bacteroidia, Clostridia, Gammaproteobacteria, and Bacilli.

A method for medical treatment is disclosed. The method comprises: administering a microbiota restoration therapy composition to a patient with a medical disorder; obtaining a fecal sample from the patient at least 7 days after administering the microbiota restoration therapy composition to the patient; calculating a microbiome health index based on the relative abundance of bacteria from a selected group of taxonomic classes in order to assess the success of treating the patient; and wherein the selected group of taxonomic classes include Bacteroidia, Clostridia, Gammaproteobacteria, and Bacilli.

Alternatively or additionally to any of the embodiments above, when the calculated microbiome health index is lower than a pre-determined threshold value, administering a second microbiota restoration therapy composition to the patient.

Alternatively or additionally to any of the embodiments above, the method for medical treatment further comprises obtaining a pre-treatment fecal sample from the patient prior to the treatment and calculating another microbiome health index based on the relative abundance of bacteria from the selected group of taxonomic classes in the pre-treatment fecal sample.

Alternatively or additionally to any of the embodiments above, when the patient's microbiome health index in the post-treatment fecal sample is about the same or not substantially higher (for example, less than one, two, three, or four order of magnitude) than the microbiome health index in the pre-treatment fecal sample, administering a second microbiota restoration therapy composition to the patient.

Alternatively or additionally to any of the embodiments above, the microbiota restoration therapy composition has a microbiome health index (calculated based on the relative abundance of bacteria from the selected group of taxonomic classes in the composition) that is higher (e.g., at least 10 times, 15 times, 20 times, 30 times, 40 times, 50 times, or 100 times higher) than the microbiome health index of the pre-treatment fecal sample from the patient.

Alternatively or additionally to any of the embodiments above, the microbiota restoration therapy composition has a microbiome health index (calculated based on the relative abundance of bacteria from the selected group of taxonomic classes in the composition) that is higher (e.g., at least 10 times, 15 times, 20 times, 30 times, 40 times, 50 times, or 100 times higher) than the microbiome health index of the post-treatment fecal sample from the patient.

A method for assessing microbiota is disclosed. The method comprises: obtaining a fecal sample from a patient; calculating a microbiome health index based on the relative abundance of bacteria from one or more taxonomic classification levels in the fecal sample; and correlating the microbiome health index with a medical condition of the patient.

Alternatively or additionally to any of the embodiments above, correlating the microbiome health index with a medical condition of the patient may include determining whether or not the microbiome health index is below a pre-determined threshold.

Alternatively or additionally to any of the embodiments above, determining whether or not the microbiome health index is below a pre-determined threshold may include identifying the patient as having an unbalanced/unhealthy microbiome.

Alternatively or additionally to any of the embodiments above, correlating the microbiome health index with a medical condition of the patient may include determining whether or not the microbiome health index is above a pre-determined threshold.

Alternatively or additionally to any of the embodiments above, determining whether or not the microbiome health index is above a pre-determined threshold may include identifying the patient as having a balanced/healthy microbiome.

Alternatively or additionally to any of the embodiments above, calculating a microbiome health index based on the relative abundance of bacteria from one or more taxonomic classification levels in the fecal sample includes calculating the microbiome health index based on the relative abundance of bacteria from one or more taxonomic phyla.

Alternatively or additionally to any of the embodiments above, calculating a microbiome health index based on the relative abundance of bacteria from one or more taxonomic classification levels in the fecal sample includes calculating the microbiome health index based on the relative abundance of bacteria from one or more taxonomic classes.

Alternatively or additionally to any of the embodiments above, calculating a microbiome health index based on the relative abundance of bacteria from one or more taxonomic classification levels in the fecal sample includes calculating the microbiome health index based on the relative abundance of bacteria from one or more taxonomic orders.

Alternatively or additionally to any of the embodiments above, calculating a microbiome health index based on the relative abundance of bacteria from one or more taxonomic classification levels in the fecal sample includes calculating the microbiome health index based on the relative abundance of bacteria from one or more taxonomic families.

Alternatively or additionally to any of the embodiments above, calculating a microbiome health index based on the relative abundance of bacteria from one or more taxonomic classification levels in the fecal sample includes calculating the microbiome health index based on the relative abundance of bacteria from one or more taxonomic genera.

Alternatively or additionally to any of the embodiments above, calculating a microbiome health index based on the relative abundance of bacteria from one or more taxonomic classification levels in the fecal sample includes calculating the microbiome health index based on the relative abundance of bacteria from one or more taxonomic species.

A method for assessing a medical treatment is disclosed. The method comprises: treating a patient for a medical disorder; a pre-determined time period after treating the patient for the medical disorder, obtaining a fecal sample from a patient; calculating a microbiome health index based on the relative abundance of bacteria from one or more taxonomic classification levels in the fecal sample; and determining the success of treating the patient for the medical disorder.

Alternatively or additionally to any of the embodiments above, calculating a microbiome health index based on the relative abundance of bacteria from one or more taxonomic classification levels in the fecal sample includes calculating the microbiome health index based on the relative abundance of bacteria from one or more taxonomic phyla.

Alternatively or additionally to any of the embodiments above, calculating a microbiome health index based on the relative abundance of bacteria from one or more taxonomic classification levels in the fecal sample includes calculating the microbiome health index based on the relative abundance of bacteria from one or more taxonomic classes.

Alternatively or additionally to any of the embodiments above, calculating a microbiome health index based on the relative abundance of bacteria from one or more taxonomic classification levels in the fecal sample includes calculating the microbiome health index based on the relative abundance of bacteria from one or more taxonomic orders.

Alternatively or additionally to any of the embodiments above, calculating a microbiome health index based on the relative abundance of bacteria from one or more taxonomic classification levels in the fecal sample includes calculating the microbiome health index based on the relative abundance of bacteria from one or more taxonomic families.

Alternatively or additionally to any of the embodiments above, calculating a microbiome health index based on the relative abundance of bacteria from one or more taxonomic classification levels in the fecal sample includes calculating the microbiome health index based on the relative abundance of bacteria from one or more taxonomic genera.

Alternatively or additionally to any of the embodiments above, calculating a microbiome health index based on the relative abundance of bacteria from one or more taxonomic classification levels in the fecal sample includes calculating the microbiome health index based on the relative abundance of bacteria from one or more taxonomic species.

The above summary of some embodiments is not intended to describe each disclosed embodiment or every implementation of the present disclosure. The Detailed Description, which follows, more particularly exemplifies these embodiments.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The intestinal microbiota (e.g., a collection of microorganisms including, but not limited to, bacteria, fungi, phages, viruses, etc., and the like) within/along the gastrointestinal tract, and/or the disruption thereof, may be considered to be correlated with a number of conditions. Some of these conditions are closely associated with the digestive tract such as Clostridium difficile infection, ulcerative colitis, inflammatory bowel disease, dysbiosis, other disorders and/or diseases of the digestive tract, cancer (e.g., along the digestive tract), and/or the like. Other conditions that may be correlated with the intestinal microbiota may include diabetes, cancer (e.g., including cancer at a location other than along the digestive tract), autism, hepatic encephalopathy, etc. These examples are meant to be illustrative, not comprehensive.

Because of the sheer number of organisms and/or the diversity among organisms that make up the microbiota, it may be challenging to characterize the microbiota in a manner that allows for a clinician to assess changes to the microbiota, assess/diagnose a medical condition, assess a medical treatment, or otherwise correlate the microbiota with a clinically meaningful condition. Disclosed herein are methods for characterizing the intestinal microbiota. The methods may allow a clinician to assess a medical condition, diagnose a medical condition/disorder, evaluate the success of a medical treatment, combinations thereof, and the like, and/or otherwise provide clinically meaningful information to a clinician.

In at least some instances, an index such as a unidimensional index termed the Microbiome Health Index (MHI) may be calculated/determined for the intestinal microbiota of a patient. The MHI may be determined based on the relative abundance of bacteria from one or more taxonomic classification levels (e.g., phylum, class, order, family, genus, species, sub-species, etc.) in the intestinal microbiota. The relative abundance of such bacteria can be quantified and used in a calculation that determines the MHI. For example, the MHI may be determined based on the relative abundance of bacteria from a selected group of taxonomic classes (e.g., one or more classes) present in the intestinal microbiota and the relative abundance of such bacteria can be quantified and used in a calculation that determines the MHI. The magnitude of the MHI, once calculated/determined, may be correlated with a medical condition, used to assess the health of a patient, used to assess the success of the medical treatment, used to characterize the microbiota, and/or other which provide clinically relevant information.

In at least some instances, calculating/determining the MHI may include collecting a fecal sample from a patient and determining/quantifying the relative abundance of bacteria (e.g., quantifying the relative abundance of bacteria from selected taxonomic classes) in the fecal sample. Determining/quantifying the relative abundance of bacteria from selected taxonomic classes may include the use of a suitable methodology such as 16s rRNA or whole genome sequencing. Other methods are contemplated.

Calculating/determining the MHI may also include determining/quantifying a first sum of relative bacterial abundance. The first sum of bacterial abundance (e.g., relative to the entire population of bacteria in the fecal sample) may be understood to be or otherwise be equal to the sum of a first relative abundance of bacteria from a taxonomic classification level (e.g., phylum, class, order, family, genus, species, sub-species, etc.) and a second relative abundance of bacteria from the taxonomic classification level (e.g., phylum, class, order, family, genus, species, sub-species, etc.). For example, the first sum of bacterial abundance may be equal to the sum of a first relative abundance of a first taxonomic class of bacteria and a second relative abundance of a second taxonomic class of bacteria. In this example, the first sum of bacterial abundance may include the quantification of the relative abundance of bacteria from two taxonomic classes. However, this is not intended to be limiting. In other instances, the first sum of bacterial abundance may include the quantification of the relative abundance of bacteria from more or fewer than two taxonomic classes of bacteria. Furthermore, in the above example, all of the bacteria that make up the first sum of bacterial abundance are from the same taxonomic classification level (e.g., class). This is not intended to be limiting. The first sum of bacterial abundance may include the quantification of the relative abundance of bacteria from different taxonomic classification levels.

Calculating/determining the MHI may also include determining/quantifying a second sum of relative bacterial abundance. The second sum of bacterial abundance (e.g., relative to the entire population of bacteria in the fecal sample) may be understood to be or otherwise be equal to the sum of a third relative abundance of bacteria from the taxonomic classification level (e.g., phylum, class, order, family, genus, species, sub-species, etc.) and a fourth relative abundance of bacteria from the taxonomic classification level (e.g., phylum, class, order, family, genus, species, sub-species, etc.). For example, the second sum of bacterial abundance may be understood to be or otherwise be equal to the sum of a third relative abundance of a third taxonomic class of bacteria and a fourth relative abundance of a fourth taxonomic class of bacteria. Just like the prior example, the second sum of bacterial abundance may include the quantification of the relative abundance of bacteria from two taxonomic classes. However, this is not intended to be limiting. In other instances, the second sum of bacterial abundance may include the quantification of the relative abundance of bacteria from more or fewer than two taxonomic classes of bacteria. Furthermore, the second sum of bacterial abundance may include the quantification of the relative abundance of bacteria from the same or different taxonomic classification levels.

Finally, calculating the MHI may include dividing the first sum of relative bacterial abundance by the second sum of relative bacterial abundance.

Increased levels of some taxonomic classes of bacteria may be associated with a healthy or improving microbiota. For example, the relative abundance of bacteria from the taxonomic classes Bacteroidia and/or Clostridia may be correlated with successful treatment of a number of digestive conditions. Conversely, elevated levels of bacteria from the taxonomic classes Gammaproteobacteria and/or Bacilli may be associated with microbiota disruption or dysbiosis. Based on these observations, in at least some instances, calculating/determining the MHI may include determining/quantifying a first sum of relative bacterial abundance (e.g., in a fecal sample) that is equal to the relative abundance of bacteria from the taxonomic class Bacteroidia added to the relative abundance of bacteria from the taxonomic class Clostridia. Calculating/determining the MHI may also include determining/quantifying a second sum of relative bacterial abundance that is equal to the relative abundance of bacteria from the taxonomic class Gammaproteobacteria added to the relative abundance of bacteria from the taxonomic class Bacilli. Finally, calculating the MHI may include dividing the first sum of relative bacterial abundance by the second sum of relative bacterial abundance.

The result of calculating the MHI is a value (e.g., a number). In at least some instances, the magnitude of the MHI may be used to assess a medical condition, diagnose a medical condition/disorder, evaluate the success of a medical treatment, combinations thereof, and the like, and/or otherwise provide clinically meaningful information to a clinician. For example, if the magnitude of the MHI is below a pre-determined threshold value, a clinician may assess/diagnose the microbiota as unbalanced, unhealthy, or the like. In some instances, the threshold value may be on the order of about 1-20, or about 1-10, or about 5-10, or about 5-8, or about 7-7.2, or about 7.1. For example, if the pre-determined threshold value is set at 7.1, a fecal sample with a calculated MHI of 1 (e.g., below the pre-determined threshold) would be considered to be correlated with and/or identified as an unbalanced/unhealthy microbiome whereas a fecal sample with a calculated MHI of 10 (e.g., above the pre-determined threshold) would be considered to be correlated with and/or identified as a balanced/healthy microbiome. Thus, the MHI may be used to clinically evaluate or determine if a patient has a heathy or unhealthy microbiome and this evaluation may be used by the clinician to determine an appropriate intervention/treatment for the patient, as desired. In other instances, the threshold value may be on the order of about 1-20, or about 1-10, or about 5-10, or about 6-9, or about 8-8.5, or about 8.2. For example, if the pre-determined threshold value is set at 8.2, a fecal sample with a calculated MHI of 1 (e.g., below the pre-determined threshold) would be considered to be correlated with and/or identified as an unbalanced/unhealthy microbiome whereas a fecal sample with a calculated MHI of 10 (e.g., above the pre-determined threshold) would be considered to be correlated with and/or identified as a balanced/healthy microbiome. In other instances, the threshold value may be on the order of about 1-20, or about 1-10, or about 5-10, or about 6-9, or about 8-8.5, or about 8.4. For example, if the pre-determined threshold value is set at 8.2, a fecal sample with a calculated MHI of 1 (e.g., below the pre-determined threshold) would be considered to be correlated with and/or identified as an unbalanced/unhealthy microbiome whereas a fecal sample with a calculated MHI of 10 (e.g., above the pre-determined threshold) would be considered to be correlated with and/or identified as a balanced/healthy microbiome. In other instances, the threshold value may be on the order of about 1-50, or about 1-40, or about 5-40, or about 30-35, or about 31. For example, if the pre-determined threshold value is set at 31, a fecal sample with a calculated MHI of 1 (e.g., below the pre-determined threshold) would be considered to be correlated with and/or identified as an unbalanced/unhealthy microbiome whereas a fecal sample with a calculated MI-H of 40 (e.g., above the pre-determined threshold) would be considered to be correlated with and/or identified as a balanced/healthy microbiome. Thus, the MHI may be used to clinically evaluate or determine if a patient has a heathy or unhealthy microbiome and this evaluation may be used by the clinician to determine an appropriate intervention/treatment for the patient, as desired.

In some instances, the MHI may be used to assess the success of a medical treatment. For example, in a patient suffering a gastrointestinal disorder such as a Clostridium difficile infection, the patient may be treated with a microbiota restoration therapy composition (e.g., such as those described/disclosed in U.S. Pat. Nos. 9,629,881, 9,675,648, U.S. Patent Application Pub. No. US 2016/0361263, and U.S. patent application Ser. No. 16/009,157, the entire disclosures of which are herein incorporated by reference). At one or more time periods after treatment, a fecal sample may be collected from the patient and the MHI may be calculated. If the magnitude of the MHI is below a pre-determined threshold value (e.g., such as those disclosed herein), a clinician may determine whether or not the treatment was successful. For example, if the pre-determined threshold value is set at 8.2, a fecal sample (collected at a suitable time period after treatment) with a calculated MI-II of 1 (e.g., below the pre-determined threshold) would indicate that the treatment was unsuccessful whereas a fecal sample (collected at a suitable time period after treatment) with a calculated MHI of 10 (e.g., above the pre-determined threshold) would indicate that the treatment was successful. In some instances, the time period (e.g., the number of days following treatment with a microbiota restoration therapy composition) may be on the order of about 1-60 days, or about 7-60 days, or about 7-30 days, or about 7-15 days, or about 7 days, or more than about 7 days after treatment.

Alternatively or additionally, a number of methods are contemplated. Some of these methods may include assessing the microbiota of a patient to determine if the patient is “healthy” (e.g., has intestinal microbiota consistent with other individuals considered to be healthy (e.g., without disease or illness)) or is “unhealthy” (e.g., has intestinal microbiota consistent with other individuals considered to be “unhealthy” (e.g., with one or more diseases or illnesses). This may include collecting a fecal sample from the patient and determining the MHI. If the MHI is below a pre-determined threshold, the patient may be considered “unhealthy” or to have a disease or illness and may be treated with a microbiota restoration therapy composition. If the MHI is above a pre-determined threshold, no further treatment may be need.

Alternatively or additionally, a number of methods are contemplated. Some of these methods may include assessing the microbiota of a patient over time. This may include collecting a fecal sample from the patient at one or more time periods and determining the MHI at the time periods. Variation in the MHI over time can be correlated with a medical condition.

Alternatively or additionally, a number of methods are contemplated. Some of these methods may include manufacturing a microbiota restoration therapy composition. Manufacturing a microbiota restoration therapy composition may include collecting a fresh human fecal sample, adding a diluent to the fresh human fecal sample to form a diluted sample, and filtering the diluted sample to form a filtrate comprising the microbiota restoration therapy composition. In some instances, the diluent may include 30-90 g/L polyethylene glycol in saline. In some of these and in other instances, the MHI may be measured in a fecal sample of a patient with a medical disorder. In some of these and in other instances, the patient may be treated with the microbiota restoration therapy composition. In some of these and in other instances, the MHI may be measured in a fecal sample of the patient at various time periods after treatment (e.g., 7 days after treatment, 30 days after treatment, 60 days after treatment, etc.). If the MHI is below a pre-determined threshold, the patient may be treated a second time with a microbiota restoration therapy composition (e.g., that may be derived from the same or a different sample, from the same donor or a different donor, etc.). In some of these and in other instances, the MHI may be measured in a fecal sample of the patient at various time periods after the second treatment (e.g., 7 days after treatment, 30 days after treatment, 60 days after treatment, etc.).

### EXAMPLES

The disclosure may be further clarified by reference to the following Examples, which serve to exemplify some embodiments, and not to limit the disclosure in any way.

### Example 1—MHI can Distinguish Between “Healthy” and “Unhealthy” Microbiomes

The MHI was calculated for: (Group A) fecal samples collected from patients diagnosed with a Clostridium difficile infection who had received an antibiotic therapy, (Group B) fecal samples collected from healthy individuals and then processed into a microbiota restoration therapy composition known as RBX2660 manufactured by Rebiotix, Inc., and (Group C) a fecal transplant material prepared under the protocols of the Human Microbiome Project. In this example, MHI was calculated using Equation 1 below:

MHI=(RA_{Bacteroidia}+RA_{Clostridia})/(RA_{Gammaproteobacteria}+RA_{Bacilli}) Equation 1:

where:
RA_{Bacteroidia} is the relative abundance of bacteria from the taxonomic class Bacteroidia,
RA_{Clostridia} is the relative abundance of bacteria from the taxonomic class Clostridia,
RA_{Gammaproteobacteria} is the relative abundance of bacteria from the taxonomic class Gammaproteobacteria,
RA_{Bacilli} is the relative abundance of bacteria from the taxonomic class Bacilli.

The relative abundance values were calculated using 16s rRNA analysis using the Illumina MiSeq platform. Group A was classified as “unhealthy” whereas Group B and Group C were classified as “healthy”. The results indicated that the MHI values from Group A had a mean of 0.78 and median of 0.002, the MHI values from Group B had a mean of 260 and median of 200, and the MHI values from Group C had a mean of 1500 and median of 868. A statistically significant cutoff point for distinguishing “unhealthy” and “healthy” MHI was determined to be 7.1 (sensitivity=0.96, specificity=0.99, likelihood ratio=0.8).

### Example 2—MHI Differs Among Successful and Failed Treatment Responses Following Treatment with a Microbiota Restoration Therapy Composition Known as RBX2660 Manufactured by Rebiotix, Inc

The MHI was calculated using Equation 1 based on fecal samples collected from patients diagnosed with a Clostridium difficile infection (and had received an antibiotic treatment) prior to treatment with a microbiota restoration therapy composition known as RBX2660 manufactured by Rebiotix, Inc. Fecal samples were again collected from the patients with a Clostridium difficile infection (and had received an antibiotic treatment) 7 days after treatment, 30 days after treatment, and 60 days after treatment with a microbiota restoration therapy composition known as RBX2660 manufactured by Rebiotix, Inc. The results of the MHI determinations were compared in patient groups where the treatment was clinically determined to be successful and in patient groups where the treatment was clinically determined to be a failure. The results are summarized in Table 1.

TABLE 1
MHI Before and After Treatment

| | | | Fraction of | |
|---|---|---|---|---|
| | | | patients with a | |
| Group | n | Median MHI | MHI > 7.1 | P vs BL |
| BL | 47 | 0.0018 | 0.04 | <0.001 |
| 7 d success | 35 | 9.4 | 0.51 | <0.001 |
| 30 d success | 27 | 16 | 0.67 | <0.001 |
| 60 d success | 27 | 61 | 0.74 | <0.001 |
| 7 d failure | 13 | 1.7 | 0.23 | 0.057 |
| 30 d failure | 5 | 0.47 | 0.20 | n/a |
| RBX2660 | 84 | 182 | 0.99 | <0.001 |

BL = baseline, patients diagnosed with a Clostridium difficile infection prior to treatment with a microbiota restoration therapy composition. RBX2660 = a microbiota restoration therapy composition manufactured by Rebiotix, Inc.

### Example 3—Successful Patients Show Continual MHI Increase

MHI in fecal samples from patients diagnosed with a Clostridium difficile infection was determined using Equation 1 at a number of time periods after treatment with a microbiota restoration therapy composition known as RBX2660 manufactured by Rebiotix, Inc. In patients that were clinically determined to have a successful treatment, the value of MHI increased continually when measured 7 days post treatment, 30 days post treatment, and 60 days post treatment (p<0.001 by Sign pairwise test compared to baseline). In patients that were clinically determined to have a failed treatment, the value of MHI did not continually increase when measured 7 days post treatment and 30 days post treatment (MHI at 7 days post treatment was not significantly different among successes and failures; p=0.24, Kolmagorov-Smirnov test).

### Example 4—Success MHI Approaches RBX2660 MHI

Receiver Operating Characteristic (ROC) analysis showed that the ability of MHI to distinguish between responders (e.g., patients deemed clinically to have been successfully treated by a microbiota restoration therapy composition known as RBX2660 manufactured by Rebiotix, Inc.) and RBX2660 diminished with increasing time after treatment.

### Example 5—MHI Significantly Increased Among Responders in the PUNCH Open-Label Trial—Comparison with the PUNCH CD2 Trial

The MHI was calculated using Equation 1 based on fecal samples collected from patients diagnosed with a Clostridium difficile infection (and had received an antibiotic treatment) prior to treatment with a microbiota restoration therapy composition known as RBX2660 manufactured by Rebiotix, Inc. Fecal samples were again collected from the patients with a Clostridium difficile infection (and had received an antibiotic treatment) 7 days after treatment and 30 days after treatment with a microbiota restoration therapy composition known as RBX2660 manufactured by Rebiotix, Inc. In this experiment, the data from Example 2 is updated with additional patient results (PUNCH CD2 trial). In addition, the results from the PUNCH Open-Label trial are also included as well as pooled results (where data from the PUNCH CD2 trial and the PUNCH Open-Label trial are combined/pooled). The results of the MHI determinations were compared in patient groups where the treatment was clinically determined to be successful and in patient groups where the treatment was clinically determined to be a failure. The results are summarized in Table 2.

TABLE 2
MHI Before and After Treatment

| | | | | Fraction of | |
|---|---|---|---|---|---|
| | | | | Samples with | Comparison |
| Sample group | | Median MHI (CI) | | MHI > 7.4 | to baseline |
| Baseline | CD 2 | 0.002 | (0.0012-0.0033) | 0.03 | NA |
| | Open-Label | 0.003 | (0.0012-0.0063) | 0.05 | NA |
| | Pooled | 0.0024 | (0.0016-0.0042) | 0.04 | NA |
| | Responders | 0.0018 | (0.0012-0.0026) | 0.04 | NA |
| | Non- | 0.0107 | (0.0025-0.0202) | 0.05 | NA |
| | responders | | | | |
| 7 days | Responders | 15.6 | (4.64-31.5) | 0.59 | <0.0001 |
| | Non- | 1.67 | (0.44-4.52) | 0.20 | 0.0008 |
| | responders | | | | |
| 30 days | Responders | 14.6 | (8.79-26.4) | 0.65 | <0.0001 |
| | Non- | 0.75 | (0.023-25.0) | 0.38 | 0.003 |
| | responders | | | | |
| RBX2660 | CD 2 | 183 | (121-276) | 0.99 | <0.0001 |
| | Open-Label | 523 | (375-579) | 1.0 | <0.0001 |
| | Pooled | 308 | (207-365) | 0.99 | <0.0001 |

Receiver Operating Characteristic (ROC) analysis was conducted for baseline versus RBX2660 samples, with MHIs from both trials pooled to maximize the population on which subsequent development was based. ROC analysis examines the diagnostic ability of a binary classifier system as its discrimination cutpoint is varied, with a higher area under the curve (AUC) indicating a diagnostic or biomarker that is more discriminatory between the two populations analyzed (Grund and Sabin, 2010). The ROC analysis indicates that the MHI was highly effective at distinguishing dysbiotic recipient microbiomes from healthier donor microbiomes (AUC=0.996; FIG. 2C). Using the ROC analysis, an MHI value of 7.4 was determined as an optimal efficacy cutpoint (sensitivity=0.96, specificity=0.99, likelihood ratio=125) for the pooled data. Subsequent analysis confirmed that considering the two RBX2660 and baseline populations separately or jointly gave similar analytical values and predictive capability (p=0.335). It is noted that the optimal efficacy cutpoint for the PUNCH CD2 trial was determined to be 8.2 (e.g., from Example 5) and the optimal efficacy cutpoint for PUNCH Open-Label trial was determined to be 31.

The median baseline MHI values for the PUNCH CD 2 and PUNCH Open-Label trials were 0.002 (0.0012−0.0033, lower and upper confidence intervals) and 0.003 (0.0012−0.0063), respectively (FIG. 2A, Table 2). The similarity of baseline MHI values between the trials is notable because the samples from the PUNCH CD 2 trial were sequenced with a 16S method whereas, the samples from PUNCH Open-Label trial were sequenced with a shallow-shotgun method. The similarity of results suggests that reliable MHI calculations can be obtained regardless of what sequencing method is used.

### Example 6—Treatment/Prevention of rCDI with RBX7455

Patients were screened and then enrolled in a phase 1 clinical trial for use of RBX7455 (an oral microbiota restoration therapy composition as disclosed herein and/or in U.S. Patent Application Pub. No. US 2016/0361263 and/or U.S. patent application Ser. No. 16/009,157, the entire contents of which are herein incorporated by reference) to prevent recurrent Clostridium difficile infection (rCDI). Following an antibiotic washout period of 24-48 hour, patients were treated. The MHI at baseline for RBX7455 participants (0.0095) was similar to RBX2660 participants at baseline (0.002; see, for example, Example 5). The MHI, 30 days after successful RBX7455 treatment is 33.3 (e.g., greater than 8.2) and consistent with RBX2660 participants (RBX2660 participants had an MHI 30 days after successful treatment of 14.6, see Example 5). RBX7455 was determined to have a median MHI 115 (mean was 109).

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

1-44. (canceled)

45. A method for assessing microbiota, the method comprising:
obtaining a fecal sample from a patient;
quantifying the relative abundance of bacteria from a selected group of taxonomic classes in the fecal sample;
calculating a microbiome health index based on the relative abundance of bacteria from the selected group of taxonomic classes; and
correlating the microbiome health index with a medical condition of the patient.

46. The method of claim 45, wherein the selected group of taxonomic classes includes bacteria from the class Bacteroidia.

47. The method of claim 45, wherein the selected group of taxonomic classes includes bacteria from the class Clostridia.

48. The method of claim 45, wherein the selected group of taxonomic classes includes bacteria from the class Gammaproteobacteria.

49. The method of claim 45, wherein the selected group of taxonomic classes includes bacteria from the class Bacilli.

50. The method of claim 45, wherein calculating a microbiome health index includes determining a first sum of relative bacterial abundance, the first sum of bacterial abundance being equal to the sum of a first relative abundance of a first taxonomic class of bacteria and a second relative abundance of a second taxonomic class of bacteria;
determining a second sum of relative bacterial abundance, the second sum of bacterial abundance being equal to the sum of a third relative abundance of a third taxonomic class of bacteria and a fourth relative abundance of a fourth taxonomic class of bacteria; and
dividing the first sum of relative bacterial abundance by the second sum of relative bacterial abundance.

51. The method of claim 50, wherein the first relative abundance of bacteria is the relative abundance of bacteria from the taxonomic class Bacteroidia in the fecal sample.

52. The method of claim 51, wherein the second relative abundance of bacteria is the relative abundance of bacteria from the taxonomic class Clostridia in the fecal sample.

53. The method of claim 52, wherein the third relative abundance of bacteria is the relative abundance of bacteria from the taxonomic class Gammaproteobacteria in the fecal sample.

54. The method of claim 53, wherein the fourth relative abundance of bacteria is the relative abundance of bacteria from the taxonomic class Bacilli in the fecal sample.

55. A method for assessing a medical treatment, the method comprising:
treating a patient for a medical disorder;
a pre-determined time period after treating the patient for the medical disorder, obtaining a fecal sample from a patient;
quantifying the relative abundance of bacteria from a selected group of taxonomic classes in the fecal sample;
calculating a microbiome health index based on the relative abundance of bacteria from the selected group of taxonomic classes; and
determining the success of treating the patient for the medical disorder.

56. The method of claim 55, wherein calculating a microbiome health index includes determining a first sum of relative bacterial abundance, the first sum of bacterial abundance being equal to the sum of a first relative abundance of a first taxonomic class of bacteria and a second relative abundance of a second taxonomic class of bacteria;
determining a second sum of relative bacterial abundance, the second sum of bacterial abundance being equal to the sum of a third relative abundance of a third taxonomic class of bacteria and a fourth relative abundance of a fourth taxonomic class of bacteria; and
dividing the first sum of relative bacterial abundance by the second sum of relative bacterial abundance.

57. The method of claim 56, wherein the first relative abundance of bacteria is the relative abundance of bacteria from the taxonomic class Bacteroidia in the fecal sample.

58. The method of claim 57, wherein the second relative abundance of bacteria is the relative abundance of bacteria from the taxonomic class Clostridia in the fecal sample.

59. The method of claim 58, wherein the third relative abundance of bacteria is the relative abundance of bacteria from the taxonomic class Gammaproteobacteria in the fecal sample.

60. The method of claim 59, wherein the fourth relative abundance of bacteria is the relative abundance of bacteria from the taxonomic class Bacilli in the fecal sample.

61. The method of claim 55, wherein the microbiota restoration therapy composition includes a processed fecal sample and a diluent including polyethylene glycol at a concentration of 30-90 g/L in saline.

62. The method of claim 55, wherein the pre-determined time period after treating the patient for the medical disorder is in the range of 7 to 30 days.

63. A method for assessing microbiota, the method comprising:
obtaining a fecal sample from a patient;
calculating a microbiome health index based on the relative abundance of bacteria from a selected group of taxonomic classes; and
correlating the microbiome health index with a medical condition of the patient.

64. The method of claim 63, wherein the selected group of taxonomic classes include Bacteroidia, Clostridia, Gammaproteobacteria, and Bacilli.
